(19) United States
(12) Patent Application Publication
(10) Pub. No.: US 2022/0361909 A1
(43) Pub. Date: Nov. 17, 2022

(54) DETECTION OF A COMPLETE INCISION BY AN ULTRASONIC TREATMENT DEVICE

(71) Applicant: OLYMPUS MEDICAL SYSTEMS CORP., Tokyo (JP)

(72) Inventors: Shunsuke Matsui, Hachioji-shi (JP); Shohei Moriwaki, Akishima-shi (JP); Norihiro Yamada, Hino-shi (JP)

(73) Assignee: OLYMPUS MEDICAL SYSTEMS CORP., Tokyo (JP)

(21) Appl. No.: 17/684,225

(22) Filed: Mar. 1, 2022

Related U.S. Application Data
(60) Provisional application No. 63/187,544, filed on May 12, 2021.

(51) Int. Cl. A61B 17/32; A61B 18/14

(52) U.S. Cl. CPC A61B 17/320068; A61B 18/1445; A61B 2017/320094; A61B 2017/320095

(57) ABSTRACT

A treatment device for treating a target tissue includes a drive source having a transducer configured to convert electrical energy to mechanical vibrations, an instrument having a blade connected to the drive source and configured to apply the mechanical vibrations to the target tissue, and a control unit configured to control a supply of electrical energy to the drive source. The control unit is configured to automatically adjust the supply of electrical energy to the drive source in response to a detection of a change in a resonance frequency of the blade. The change in resonance frequency of the blade occurs after the target tissue has been completely cut by the blade.

### CROSS-REFERENCE TO RELATED APPLICATIONS

This application claims priority to and the benefit of U.S. Provisional Application No. 63/187,544, filed on May 12, 2021, which is incorporated herein by reference in its entirety.

### FIELD

The present disclosure, according to some embodiments, relates to ultrasonic treatment devices for use in cutting a target tissue. More particularly, the present disclosure relates to ultrasonic treatment devices that are configured to detect the complete incision of a target tissue. Methods for detecting the completion of an incision by an ultrasonic treatment device, and for controlling the ultrasonic treatment device in response to same are also described herein.

### BACKGROUND

Ultrasonic treatment devices are configured to utilize ultrasonic mechanical vibrations to surgically treat various medical conditions. Ultrasonic vibrations can be used, for example, to cut, dissect, and/or cauterize soft tissue of a patient. Such ultrasonic treatment devices may generally include a tissue-contacting member (which may also be referred to herein as an “end effector”) for applying ultrasonic vibrations to the tissue to be treated, an ultrasonic transducer for converting electric energy into ultrasonic vibrations, and a transmission element for transmitting the ultrasonic vibrations from the ultrasonic transducer to the tissue-contacting member. The frictional heat generated between the vibrating tissue-contacting member and the tissue is used to cut through the tissue.

In some ultrasonic treatment devices, the tissue-contacting member may be a single-component instrument, for example, a blade, ball coagulator, or hook for applying the ultrasonic vibrations to the tissue. In other ultrasonic treatment devices, the tissue-contacting member includes a multi-component instrument, for example, a grasping instrument having a blade for applying the ultrasonic vibrations to the tissue, and a jaw capable of pivoting relative to the blade such that the tissue can be clamped between the blade and the jaw. The jaw may be configured to apply a compressive force against the tissue while the ultrasonic vibrations are applied by the blade, allowing for faster cutting and/or coagulation in some instances.

The jaw of the grasping instrument may include a pad for pressing against the tissue. The pad may be composed of a polymer material, for example, polytetrafluoroethylene (PTFE) or other polymer resin, and includes a surface intended to contact the tissue when the jaw clamps against the tissue. This pad surface may also come into contact with the vibrating blade during use, for example, once the blade has completely cut through the tissue. However, this contact between the pad and the vibrating blade can result in wear or damage to the pad.

The wear of the pad can be reduced by stopping the ultrasonic vibrations once the tissue has been cut. For example, some ultrasonic devices may include a processor that is configured to stop the ultrasonic vibration in response to detecting a change in ultrasonic impedance (“US impedance”). In some such examples, during ultrasonic treatment, the tissue is denatured by frictional heat and hardened, and the US impedance increases. After the tissue has been cut, the blade and pad come into contact with each other, and the US impedance decreases. By detecting the point (peak) at which the US impedance changes from an increase to a decrease, it may be possible to detect the completion of the incision.

Depending on the type of tissue being treated, however, a “false” peak in the US impedance may occur before the actual “true” peak signifying completion of the incision. False peaks in the US impedance may occur, for example, with certain tissues having a layered structured formed of two or more layers (e.g., cervix). The false peak may be detected, for example, after only a first layer of the tissue is cut. In some such cases, when the processor detects the false peak, the processor may stop the ultrasonic vibrations prematurely before the tissue has been completely cut through, resulting in an incomplete incision.

### SUMMARY

The present disclosure, according to some embodiments, provides an ultrasonic treatment device that is configured to detect the completion of an incision of a target tissue in manner that can overcome the difficulties described above. In some embodiments, a treatment device for treating a target tissue includes a drive source having a transducer configured to convert electrical energy to mechanical vibrations, an instrument having a blade connected to the drive source and configured to apply the mechanical vibrations to the target tissue, and a control unit configured to control a supply of electrical energy to the drive source, the control unit being configured to adjust the supply of electrical energy to the drive source in response to a detection of a change in a resonance frequency of the blade. In some embodiments, the instrument comprises a jaw that is movable with respect to the blade, the instrument being configured to grasp the target tissue between the blade and a pad of the jaw. In some embodiments, the change in resonance frequency of the blade occurs when the blade contacts the pad of the jaw after the target tissue is completely cut by the blade. In some embodiments, the control unit is configured to automatically reduce or stop the supply of electrical energy to the drive source in response to the detection of the change in the resonance frequency of the blade. In some embodiments, the control unit is configured to adjust the supply of electrical energy to the drive source in response to the detection of the change in the resonance frequency in addition to a change in US impedance.

In some embodiments, the detection of the change in the resonance frequency of the blade comprises detecting a change from a first trend in the resonance frequency over time to a second trend in the resonance frequency over time. In some embodiments, the first trend is a decreasing trend in the resonance frequency, and the second trend is an increasing trend in the resonance frequency. In some embodiments, the detection of the change in the resonance frequency of the blade comprises detecting the occurrence of a peak value of the resonance frequency.

In some embodiments, the control unit is configured to determine a slope of the resonance frequency over a predetermined time period, determine a threshold value, and compare the slope to the threshold value. In some embodiments, the control unit is configured to update the slope and the threshold value every predetermined time period. In some embodiments, the threshold value is proportional to the resonance frequency at a specified time. In some embodiments, the threshold value for a given predetermined time period is a product of the resonance frequency at a start of the given predetermined time period and a coefficient. In some embodiments, the threshold value for a given predetermined time period is equal to an average resonance frequency over the given predetermined time period. In some embodiments, the threshold value for a given predetermined time period is equal to an integral of the resonance frequency over the given predetermined time period.

In some embodiments, the control unit is configured to, for each time interval in a series of time intervals: determine a slope of the resonance frequency over an immediately preceding time interval; calculate a projected value for the resonance frequency from the slope of the resonance frequency; and compare the resonance frequency to the projected value for the resonance frequency. In some embodiments, comparing the projected value for the resonance frequency to the resonance frequency comprises calculating a difference between the resonance frequency and the projected value for the resonance frequency. In some embodiments, the control unit is further configured to compare a magnitude of the difference between the resonance frequency and the projected value for the resonance frequency to a threshold value. In some embodiments, the detection of the change in the resonance frequency of the blade comprises detecting that the magnitude of the difference exceeds the threshold value. In some embodiments, if the magnitude of the difference does not exceed the threshold value within a time interval, the control unit is configured to proceed to the next time interval in the series of time intervals.

In some embodiments, a method for controlling a treatment device by using a control unit includes generating mechanical vibrations using a transducer; transmitting the mechanical vibrations to a blade connected to the transducer; measuring a resonance frequency of the blade over time; and stopping the generation of mechanical vibrations by the ultrasonic transducer in response to detecting a change in the resonance frequency of the blade by the control unit. In some embodiments, detecting the change in the resonance frequency of the blade comprises detecting, by the control unit, a change from a first trend in the resonance frequency over time to a second trend in the resonance frequency over time. In some embodiments, the first trend is a decreasing trend in the resonance frequency, and the second trend is an increasing trend in the resonance frequency. In some embodiments, detecting the change in the resonance frequency of the blade comprises detecting the occurrence of a peak value of the resonance frequency.

In some embodiments, the method further includes, for each time interval in a series of time intervals: calculating a slope of the resonance frequency over the time interval, calculating a threshold value; and comparing the slope to the threshold value. These steps may each be performed by the control unit according to some embodiments. In some embodiments, detecting the change in the resonance frequency of the blade comprises detecting that the slope exceeds the threshold value. In some embodiments, the threshold value is proportional to the resonance frequency at a specified time. In some embodiments, the threshold value for a given time interval is a product of the resonance frequency at a start of the given time interval and a coefficient. In some embodiments, the threshold value for a given time interval is equal to an average resonance frequency over the given time interval. In some embodiments, the threshold value for a given time interval is equal to an integral of the resonance frequency over the given time interval.

In some embodiments, the method further includes, for each time interval in a series of time intervals: determining a slope of the resonance frequency over an immediately preceding time interval; calculating a projected value for the resonance frequency from the slope of the resonance frequency; and comparing the resonance frequency to the projected value for the resonance frequency. In some embodiments, comparing the projected value for the resonance frequency to the resonance frequency comprises calculating a difference between the resonance frequency and the projected value for the resonance frequency. In some embodiments, the detecting the change in the resonance frequency of the blade comprises detecting that a magnitude of the difference between the resonance frequency and the projected value for the resonance frequency exceeds a threshold value. The foregoing steps may each be performed by the control unit according to some embodiments.

### BRIEF DESCRIPTION OF THE DRAWINGS

The foregoing summary, as well as the following detailed description, will be better understood when read in conjunction with the appended drawings. For the purpose of illustrating the present disclosure, there are shown in the drawings embodiments which are presently preferred, wherein like reference numerals indicate like elements throughout. It should be noted, however, that aspects of the present disclosure can be embodied in different forms and thus should not be construed as being limited to the illustrated embodiments set forth herein. The elements illustrated in the accompanying drawings are not necessarily drawn to scale, but rather, may have been exaggerated to highlight the important features of the subject matter therein. Furthermore, the drawings may have been simplified by omitting elements that are not necessarily needed for the understanding of the disclosed embodiments.

FIG. 1A is a perspective view of an example ultrasonic treatment device having a grasping instrument according to one embodiment.

FIG. 1B is an enlarged view of the grasping instrument of the ultrasonic treatment device of FIG. 1A.

FIG. 2A is a cross-sectional view illustrating a tissue being cut by a grasping instrument of an ultrasonic treatment device according to one embodiment.

FIG. 2B is a cross-sectional view showing the grasping instrument of FIG. 2A after the tissue has been cut.

FIGS. 3A and 3B are graphs respectively showing impedance over time and resonance frequency over time during operation of an ultrasonic treatment device to cut a tissue.

FIG. 3C is an enlarged portion of the graph of FIG. 3B showing a peak in the resonance frequency.

FIGS. 3D and 3E are the graphs of FIGS. 3B and 3C with further annotations to describe the changes in resonance frequency over time during operation of the ultrasonic treatment device.

FIG. 4 is a graph showing the difference between resonance frequency and projected frequency according to a method of detecting the incision of a tissue according to an example embodiment.

FIG. 5 is a diagram outlining certain steps for a method of detecting the incision of a tissue according to certain embodiments using the projected frequency.

FIG. 6 is a flow chart showing certain steps for a method of detecting the incision of a tissue according to certain embodiments, wherein a resonance frequency is compared to a projected frequency.

FIG. 7 is a graph comparing the slope of resonance frequency to a threshold according to a further example embodiment.

FIG. 8 is a graph of resonance frequency with annotations illustrating the difference in slope before and after incision of a tissue according to another example embodiment.

FIG. 9 is a flow chart showing certain steps for a method of detecting the incision of a tissue according to further embodiments, wherein a slope of the resonance frequency is compared to a threshold value that is updated at regular intervals.

FIG. 10 is a flow chart showing certain steps for a method of detecting the incision of a tissue according to further embodiments, wherein a second slope of the resonance frequency is compared to the threshold value.

FIG. 11 is a flow chart showing certain steps for a method of detecting the incision of a tissue according to further embodiments, wherein a slope of the resonance frequency is compared to a threshold value that is calculated from an average value of the resonance frequency and updated at regular intervals.

FIG. 12 is a flow chart showing certain steps for a method of detecting the incision of a tissue according to further embodiments, wherein a slope of the resonance frequency is compared to a threshold value that is calculated from an integral value of the resonance frequency and updated at regular intervals.

### DETAILED DESCRIPTION

The present subject matter will now be described more fully hereinafter with reference to the accompanying Figures, in which representative embodiments are shown. The present subject matter can, however, be embodied in different forms and should not be construed as limited to the embodiments set forth herein. Rather, these embodiments are provided to describe and enable one of skill in the art. All publications, patent applications, patents, and other references mentioned herein are incorporated by reference in their entirety.

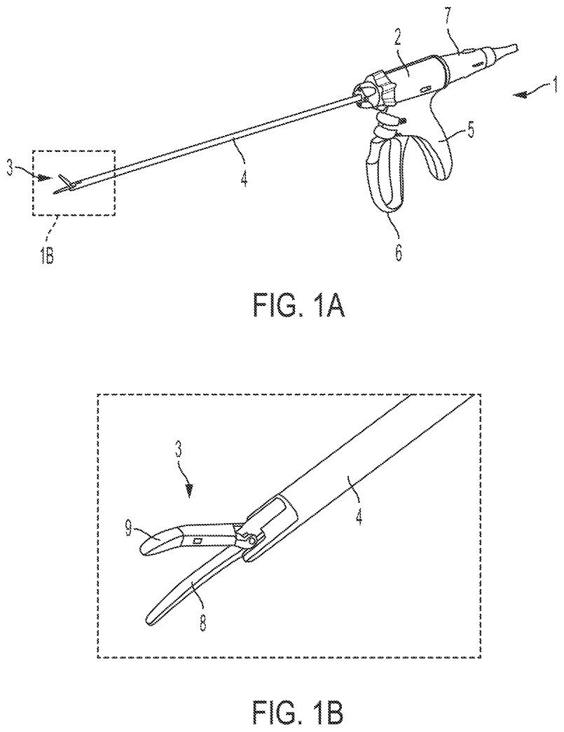

FIG. 1A provides a perspective view of an example ultrasonic treatment device 1 that, in some embodiments, includes a housing 2 and a grasping instrument 3 (which may also referred to herein as an “end effector”) located at a distal end of ultrasonic treatment device 1. Grasping instrument 3 is configured to contact and apply ultrasonic vibrations to a target tissue in order to, for example, cut and/or cauterize the target tissue. In some embodiments, grasping instrument 3 is connected to housing 2 by an elongate shaft 4. Housing 2, in some embodiments, includes a grip 5 configured to be held in the hand of a user (e.g., surgeon or other operator), and a handle 6 that is movable relative to grip 5. In some embodiments, grasping instrument 3 is configured to open and close by movement of handle 6 relative to grip 5. For example, in some embodiments, moving handle 6 away from grip 5 causes grasping instrument 3 to open, while moving handle 6 toward grip 5 causes grasping instrument 3 to close.

Ultrasonic treatment device 1 further includes a drive source 7 that includes a transducer, e.g., an ultrasonic transducer for generating the ultrasonic vibrations to be applied to a target tissue by grasping instrument 3. In some embodiments, the ultrasonic transducer is configured to convert electrical energy into mechanical vibrations, which in turn may be conducted through shaft 4 to grasping instrument 3. In some embodiments, the ultrasonic transducer of drive source 7 may include, for example, a piezoelectric element. When a voltage is applied to the piezoelectric element included in the vibrator, the piezoelectric element expands and contracts in the direction in which the voltage is applied, thus converting the electrical energy to mechanical vibrations. Housed within housing 2, according to some embodiments, is a control unit (not shown) configured to control operation of drive source 7. For example, the control unit may include one or more processors that are configured to control a supply of electrical power to the ultrasonic transducer.

FIG. 1B provides an enlarged view of grasping instrument 3, according to certain embodiments. In some embodiments, grasping instrument 3 generally includes a blade 8 (which may also be referred to herein as a “probe”) that is configured to apply ultrasonic vibrations to a target tissue, and a jaw 9 that is movable relative to blade 8 between an open configuration (an example of which is shown) and a closed configuration. Blade 8 is operatively connected to drive source 7 such that mechanical vibrations generated by the transducer are applied to the target tissue by blade 8. In use, the tissue to be treated may be clamped between blade 8 and jaw 9 such that the tissue is pressed against blade 8 by jaw 9. In some embodiments, ultrasonic vibrations generated by drive source 7 are transferred to blade 8, causing blade 8 to mechanically vibrate during use at a frequency that is sufficient for treating the target tissue (e.g., to cut the target tissue). In some embodiments, the frictional heat generated between the vibrating blade 8 and the tissue is used to cut through the tissue.

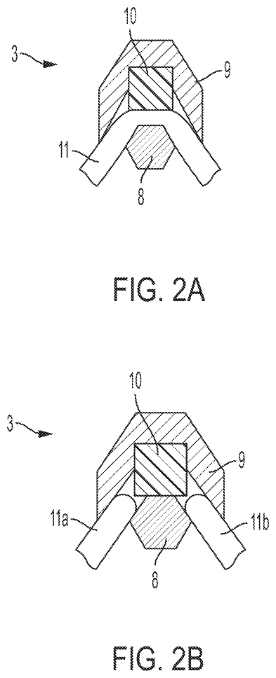

FIGS. 2A and 2B provide cross-sectional views of grasping instrument 3 during use in cutting a target tissue 11 according to some examples. As shown in FIG. 2A, target tissue 11 is positioned between and clamped by blade 8 and jaw 9 of instrument 3. In some embodiments, jaw 9 includes a pad 10 configured to abut against the target tissue 11 during use. Pad 10 may be, for example, made from a polymer or resin material, e.g., PTFE. As discussed, blade 8 is configured to vibrate at ultrasonic frequencies such that the friction between blade 8 and target tissue 11 is sufficient to cut through target tissue 11. As shown in FIG. 2B, after target tissue 11 has been cut (e.g., into pieces 11a, 11b), blade 8 may come into contact with pad 10. Contact between pad 10 and vibrating blade 8, in some instances, can result in excessive heat accumulating in pad 10 as a result of friction between the two components. This excess heat may, in turn, cause wear and/or damage to pad 10.

In some embodiments, the wear on pad 10 can be reduced by stopping the ultrasonic vibrations once the target tissue has been cut. Stopping the ultrasonic vibrations reduces the amount of friction and heat generated by the contact between blade 8 and pad 10. In some embodiments, an ultrasonic treatment device according to embodiments of the present disclosure may be configured such that the ultrasonic transducer automatically ceases operation upon detection that the tissue has been completely cut. For example, an ultrasonic treatment device according to embodiments of the present disclosure may include a control unit having one or more processors (not shown) that are configured to automatically stop the ultrasonic transducer when a complete incision in the target tissue has been detected. The control unit, for example, may be configured to automatically reduce or stop the supply of electrical energy to the transducer unit when the complete incision has been detected.

As discussed previously, US impedance can be used to determine whether a target tissue has been cut according to some examples. In some such examples, during ultrasonic treatment, the tissue is denatured by frictional heat and hardened, and the US impedance increases. After the tissue has been cut, the blade and pad come into contact with each other, and the US impedance decreases. By detecting the point (peak) at which a trend in the US impedance changes from an increase to a decrease, it may be possible to detect the completion of the incision according to some examples.

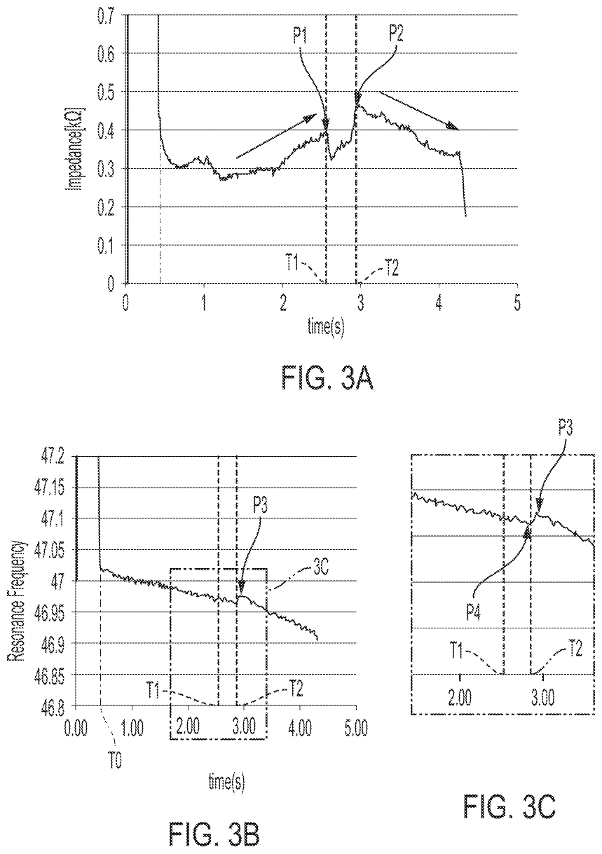

FIG. 3A is a graph showing impedance over time during the incising of a tissue by an ultrasonic treatment device according to one example. Time T0 represents the time when ultrasonic treatment was started, and time T2 represents the time when the tissue was completely cut by the ultrasonic treatment device. As shown, the impedance generally increases as the tissue is being cut up to point P2 at T2, after which the impedance generally decreases. Thus, by being able detect the occurrence of point P2 (the peak impedance), it may be possible to determine when the incision has been completed.

However, as further shown in FIG. 3A, a minor (“false”) peak impedance (point P1) may occur at a time T1 prior to reaching the “true” peak impedance (point P2) at time T2. A minor peak may occur, for example, when the target tissue being cut has a layered structure that results in a decrease in impedance after only a portion (e.g., a first layer) of the tissue is cut, but before the complete incision is made. For certain devices that are controlled based on impedance measurements, the detection of the false peak may cause the device to stop the ultrasonic vibrations prematurely before the tissue has been completely cut through, resulting in an incomplete incision.

Some embodiments of the present disclosure utilize resonance frequency and/or values derived from resonance frequency instead of or in addition to impedance to determine when a tissue has been completely cut. Resonance frequency (which may also be referred to as “resonant frequency”) is the frequency at which a system will exhibit a localized maximum response (e.g., a local maximum resonance amplitude). The resonance frequency is a unique frequency that may depend on the material, size, temperature, etc. of the blade. It has been surprisingly found that resonance frequency, in certain embodiments, can avoid the false peaks that may occur with impedance measurements, even with layered tissues. Accordingly, resonance frequency (and/or values derived from resonance frequency) may provide a more accurate detection of tissue cutting than impedance.

The resonance frequency of the vibrating blade is inversely related to the temperature of the blade. As the temperature of the blade increases, the resonance frequency of the blade decreases, and conversely, as the temperature of the blade decreases, the resonance frequency of the blade increases. In some embodiments, when the vibrating blade and the tissue come into contact, frictional heat is generated between the blade and the tissue, resulting in a decrease in the resonance frequency of the blade. When the tissue is completely cut, the blade contacts the pad provided on the jaw (as depicted in FIG. 2B). Since, in some embodiments, the pad is at a lower temperature than the blade, the temperature of the blade momentarily decreases when the blade initially contacts the pad, resulting in a momentary increase in resonance frequency. Further contact between the vibrating blade and the pad eventually causes the temperature of the blade to increase again due to the frictional heating, resulting in another decrease in resonance frequency.

In some embodiments, resonance frequency may be measured or detected by the control unit of the ultrasonic treatment device. In some embodiments, an ultrasonic treatment device may be configured to detect resonance frequency in a manner described in U.S. Pat. No. 7,983,865, which is incorporated herein by reference in its entirety. In some embodiments, the control unit may include a resonance frequency detection circuit which may be configured to detect resonance frequency based on a phase difference between the voltage and current of the ultrasonic transducer. In some embodiments, a phase of the output voltage and a phase of the output current are detected, and the phase difference between the output voltage and current is calculated. A scan is conducted to detect the resonance point (frequency) at which the phase difference between the voltage and the current is zero. In some embodiments, the control unit is configured to start the ultrasonic transducer at this detected resonance frequency.

FIGS. 3B-3E are graphs according to one example that show resonance frequency over time that was measured concurrently with the impedance measurements shown in FIG. 3A. As particularly shown in FIGS. 3D and 3E, during a first interval 101, the resonance frequency demonstrates a generally decreasing trend. During this time, which begins at time T0, the ultrasonic treatment is applied to the tissue and frictional contact between the vibrating blade of the ultrasonic treatment device and the tissue causes the temperature of the blade to increase. As discussed above, since resonance frequency is inversely related to temperature, the resonance frequency decreases as the temperature increases, resulting in the downward trend during first interval 101 shown in FIG. 3D. When the tissue is fully cut, the blade experiences a decrease in temperature, resulting in an increase in resonance frequency. This is shown during second interval 102. In some embodiments, this decrease in temperature occurs when the blade contacts the pad immediately after the tissue is cut. The pad may be at a lower temperature than the blade at that time such that the temperature of the blade decreases when contact is made between the blade and the lower temperature pad, for example, as a result of heat transfer from the blade to the pad). As shown in FIGS. 3D and 3E, second interval 102 may be substantially shorter than first interval 101. For example, first interval 101 may be more than 1-2 seconds, meanwhile second interval 102 may be less than 0.25 seconds according to some embodiments. Continued contact between the vibrating blade and the pad causes the temperature to increase again as a result of friction between the blade and the pad. This increase in temperature results in a further decrease in resonance frequency, shown in third interval 103.

The resonance frequency graphs of FIGS. 3B-3E show a single peak at point P3. Point P3 occurs at the border between second interval 102 and third interval 103, where the resonance frequency changes from an increasing trend to a decreasing trend. Point P4 occurs prior to Point P3 at the border between first interval 101 and second interval 102, where the resonance frequency changes from a decreasing trend to an increasing trend. Point P4 may represent a local minimum point in the resonance frequency. Referring particularly now to FIGS. 3B and 3C, point P4 generally coincides with time T2 at which the peak in impedance (point P2 of FIG. 3A) occurred. Accordingly, in some embodiments, detection of the point at which resonance frequency changes from a decreasing trend to an increasing trend (e.g., point P4 at the border between first interval 101 and second interval 102) may be used as an indicator to determine when the incision in the tissue has been completed as an alternative to, or in addition to, detecting the peak in impedance. In some embodiments, detection of the peak in resonance frequency (e.g., point P3) may be used as an indicator to determine when the incision in the tissue has been completed. Furthermore, unlike the impedance measurements, the resonance frequency did not exhibit a minor or “false” peak prior to point P3. For example, while the impedance graph of FIG. 3A showed a false peak (point P1) at time T1, no corresponding peak was shown in the resonance frequency graph of FIG. 3B at time T1. Use of resonance frequency can therefore avoid the problem of detecting false peaks, according to some embodiments.

Certain embodiments of the present disclosure include methods for detecting completion of an incision by an ultrasonic treatment device through the use of resonance frequency and/or values calculated or derived from the resonance frequency. In some embodiments, a method includes comparing a measured value of the resonance frequency of the blade of the ultrasonic treatment device to a projected value for the resonance frequency at regular time intervals. In some such embodiments, the projected value for the resonance frequency is calculated from a slope of the measured resonance frequency of a prior time interval. In some embodiments, a linear projection of the resonance frequency is calculated at regular time intervals, the linear projection having a slope that is equal to a slope of the measured resonance frequency of a prior (e.g., immediately previous) time interval. In some embodiments, a difference at a given time between the projected value of the resonance frequency and the measured resonance frequency is calculated, and if the value or magnitude of this difference exceeds a predetermined threshold value, it is determined that the tissue has been completely cut by the ultrasonic treatment device. In some embodiments, once the completion of the incision has been detected, the ultrasonic treatment device may be configured to automatically cease operation of the ultrasonic transducer.

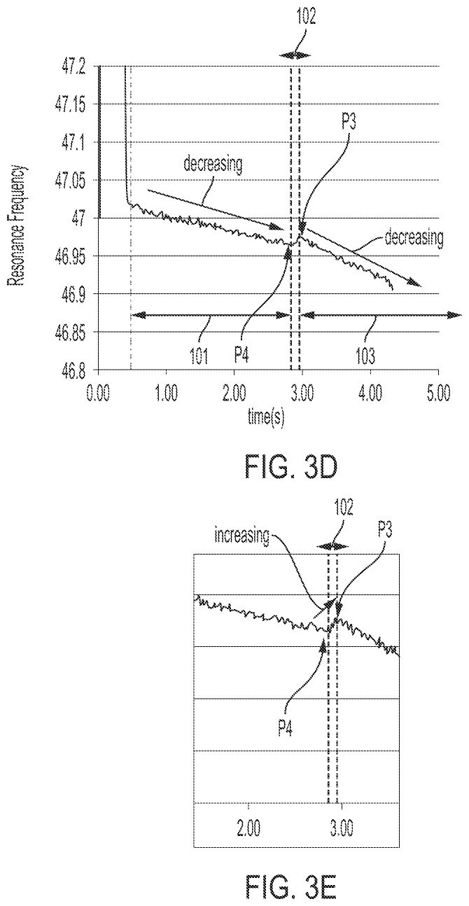

FIG. 4 shows a graph showing measured resonance frequency, projected values of the resonance frequency at regular time intervals, and the difference between the measured resonance frequency and the projected values according to one example embodiment. In this example, the projected values of the resonance frequency are linear projections calculated at regular intervals (e.g., 0.5 second intervals). In some embodiments, each linear projection has a slope that is equal to a slope calculated from the measured resonance frequency at the end points of the immediately preceding time interval. In this way, the linear projections are updated at each time interval. For example, the slope (ΔFₗᵢₙₑ) of the projected line may be calculated as the difference in the measured resonance frequency at the end points of the immediately preceding time interval of duration T1 divided by the duration of the time interval: ΔFₗᵢₙₑ=((F₀−F_{T1})/T1), where F₀ is the resonance frequency measured at the start of the time interval, and F_{T1} is the resonance frequency measured at time T1 (the end of the time interval).

In some embodiments, the difference between the projected line and the measured resonance frequency at a given time is calculated. When the value or magnitude of this difference exceeds a certain threshold value, this signifies that the measured resonance frequency has substantially diverged from the projected value for the resonance frequency. In some embodiments, this divergence indicates that the resonance frequency has reached a turning point (e.g., changed from a decreasing trend to an increasing trend), signifying that the tissue has been completely cut. The threshold value, in some embodiments, may be a predetermined constant value. In other embodiments, the threshold value may be variable. For example, the threshold value may be a function of one or more values of the measured resonance frequency, such as an initial resonance frequency.

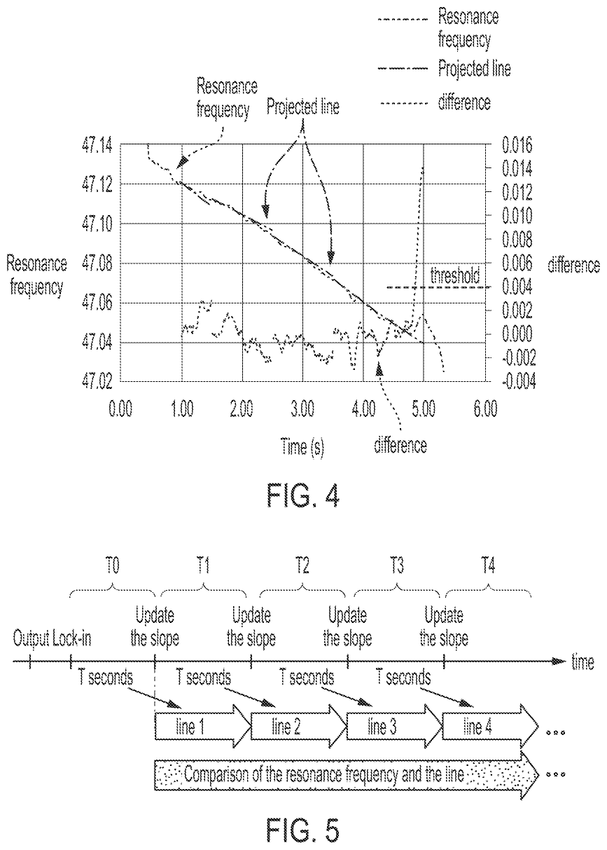

FIG. 5 is a diagram outlining the general steps of a method according to some embodiments. In some embodiments, during the output stage, a control unit of the ultrasonic treatment device drives the ultrasonic transducer and performs a scanning process to detect a resonance point (frequency) at which a phase difference between the voltage (V) and the current (I) becomes zero. When the scanning process is successful, lock-in is completed and the control unit drives the ultrasonic vibrator at the detected resonance frequency. After an initial time interval T0 of T seconds, following output and lock-in, a first line (line 1) is calculated representing the projected resonance frequencies for time interval T1. The slope of line 1 may be calculated as described above, for example, by taking the difference of the measured resonance frequency at the end points of time interval T0 divided by T seconds. Throughout time interval T1, the value of line 1 is compared to the measured resonance frequency. If, for example, the difference between the value of line 1 and the measured resonance frequency at a given time remains below a threshold value, the process continues. If the difference between the value of line 1 and the measured resonance frequency exceeds the threshold value, the process may be stopped.

After another T seconds, at the start of time interval T2, the slope is updated based on the measured resonance frequency from preceding time interval T1, and a new, second line (line 2) is calculated representing the projected resonance frequency for time interval T2. This new line (line 2) is then compared to the measured resonance frequency and the difference is again compared to the threshold value. If the threshold value has not been exceeded during interval T2, the slope is again updated at the start of time interval T3, based on the measured resonance frequency from preceding time interval T2, and a new, third line (line 3) is calculated representing the projected resonance frequency for time interval T3. Line 3 is then compared to the measured resonance frequency and the difference is again compared to the threshold value. This process may continue to repeat for every T seconds until the threshold value is exceeded.

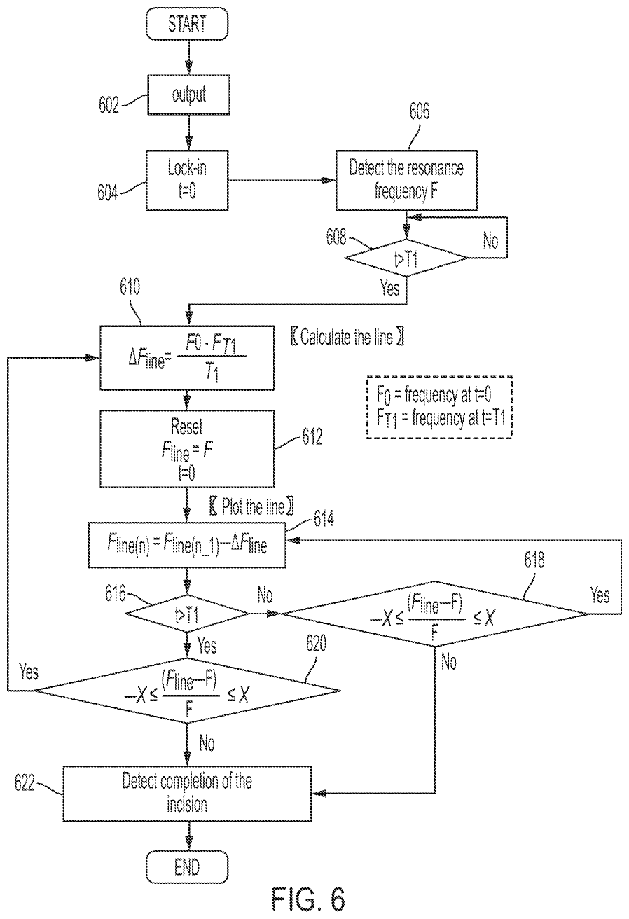

FIG. 6 is a flow chart illustrating steps for detecting completion of an incision by an ultrasonic treatment device in accordance with further embodiments. Following output step 602, time t is initialized and set to 0 at lock-in step 604. At step 606, the resonance frequency F is measured. At step 608, time t is compared to the duration T1 of a specified time interval until time t exceeds T1. The process then proceeds to step 610 where the slope ΔFₗᵢₙₑ for a projected resonance frequency line (Film) is calculated. In some embodiments, Δₗᵢₙₑ=(F₀−F_{T1})/T1, where F₀ is the resonance frequency measured at time t=0, and F_{T1} is the resonance frequency measured at time t=T1. Following the calculation of ΔFₗᵢₙₑ, the value of Fₗᵢₙₑ is set to be equal to F as an initial value and time t is reset to t=0 at reset step 612. At step 614, Fₗᵢₙₑ is calculated as Fₗᵢₙₑ₍ₙ₎=Fₗᵢₙₑ₍ₙ₋₁₎−ΔFₗᵢₙₑ, where n is the sampling interval and is less than T1.

At step 616, time t is compared to T1. If time t does not exceed T1 at step 616, the process proceeds to step 618 wherein a difference between the projected resonance frequency Fₗᵢₙₑ and the measured resonance frequency F is calculated. In some embodiments, the difference is a relative difference. In some embodiments, a relative difference (e.g., a percentage difference) between the projected resonance frequency Fₗᵢₙₑ and the measured resonance frequency F is calculated. In some such embodiments, the relative difference may be calculated by the formula (Fₗᵢₙₑ−F)/F. This relative difference, or a magnitude of the relative difference, may then be compared to a threshold value X. In some embodiments, threshold value X is a predetermined constant value. In other embodiments, threshold value X may be a function of one or more other variables or fixed parameters. In some embodiments, threshold value X may be, for example, stored in a memory or processor of the control unit. In some embodiments, if the relative difference is within the range of −X to X (e.g., −X≤(Fₗᵢₙₑ−F)/F≤X), the process returns to step 614, wherein Fₗᵢₙₑ₍ₙ₎ is recalculated for the next sampling interval. If, however, the relative difference falls outside of the range of −X to X (e.g., |(Fₗᵢₙₑ−F)/F|>X), the process proceeds to step 622 which indicates that the incision has been completed. In some embodiments, once completion of the incision has been detected, the ultrasonic treatment may automatically cease operation.

If time t exceeds T1 at step 616, the process proceeds to step 620, wherein a difference between the projected resonance frequency Fₗᵢₙₑ and the measured resonance frequency F is calculated, similar to step 618. In some embodiments, a relative difference (e.g., a percentage difference) between the projected resonance frequency Fₗᵢₙₑ and the measured resonance frequency F, which again may be calculated by the formula ((Fₗᵢₙₑ−F)/F). This relative difference may then be compared to a threshold value X. If the relative difference is within the range of −X to X (e.g., −X≤(Fₗᵢₙₑ−F)/F≤X), the process returns to step 610, wherein slope ΔFₗᵢₙₑ is recalculated for the next time interval. If the relative difference falls outside of the range of −X to X (e.g., |(Fₗᵢₙₑ−F)/F|>X), the process proceeds to step 622 which indicates that the incision has been completed. Once completion of the incision has been detected, the ultrasonic treatment may automatically cease operation.

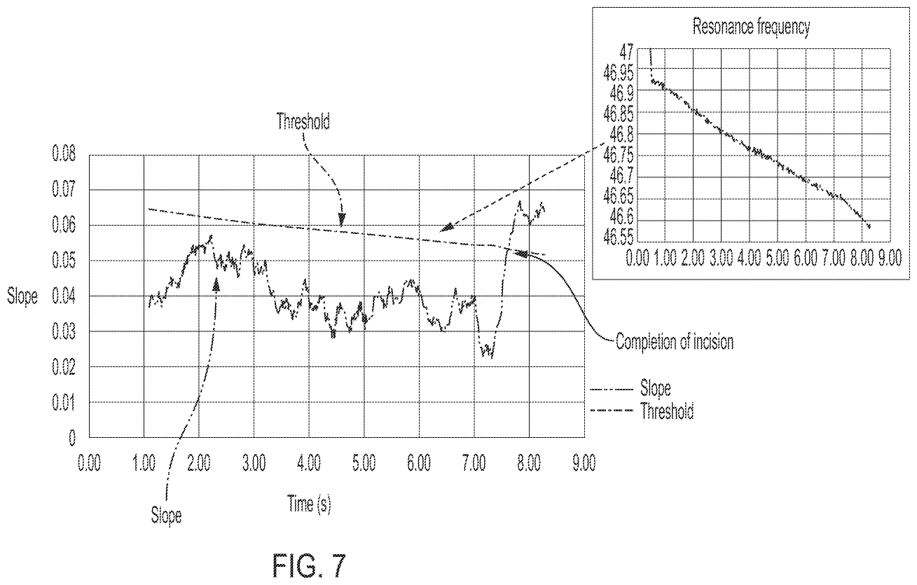

In further embodiments, detecting completion of an incision by an ultrasonic treatment device may include comparing the slope of the measured resonance frequency to a threshold value. In some embodiments, the completion of the incision is detected when the slope of the measured resonance frequency is equal to or exceeds the threshold value at a given time. In some embodiments, the slope is the first derivative of the measured resonance frequency. The slope of the measured resonance frequency may be determined, in some embodiments, at regular time intervals (e.g., every T seconds) and compared to a threshold value. In some embodiments, the threshold value may be calculated from an absolute value of the measured resonance frequency at a given time, multiplied by a coefficient. FIG. 7 is a graph showing the slope of the resonance frequency and a threshold value according to one such example. In some embodiments, the threshold value is also updated at regular time intervals (e.g., every T seconds) and may be, for example, proportional to an initial resonance frequency measured at the beginning of the time interval.

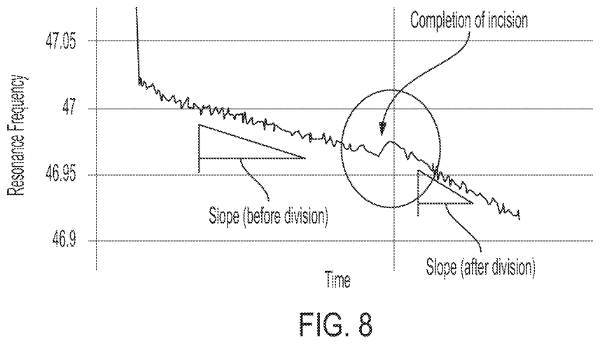

It has been found that, in some embodiments, the resonance frequency before the tissue has been fully cut may have a slope that is different than the slope of the resonance frequency after the tissue has been fully cut. This is illustrated, for example, in FIG. 8, wherein the slope of the resonance frequency after the tissue has been completely cut is steeper than the slope of the resonance frequency prior to completely cutting the tissue. In some such embodiments, the completion of the incision may be detected, in part, by comparing a first slope of the resonance frequency over a first time interval to a threshold value, and comparing a second slope of the resonance frequency over a second time interval to the threshold value. The second time interval may be less than the first time interval. In some embodiments, if the first slope exceeds the threshold value, and the second slope exceeds the threshold value for a certain period of time, the completion of the incision is indicated.

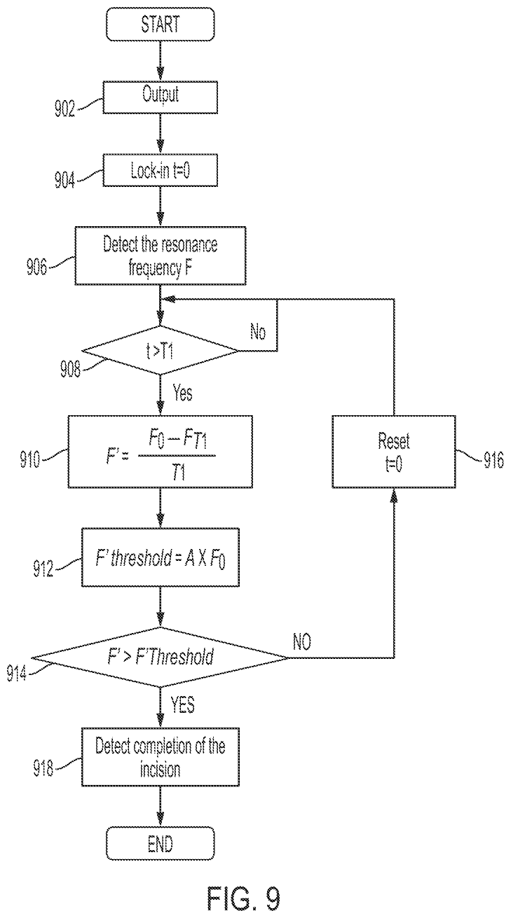

FIG. 9 is a flow chart illustrating steps for detecting completion of an incision by an ultrasonic treatment device in accordance with certain further embodiments, wherein a slope of the resonance frequency is compared to a threshold value. Following output step 902, time t is initialized and set to 0 at lock-in step 904. Steps 902 and 904 may be similar to steps 602 and 604, respectively, as described above with reference to FIG. 6. At step 906, the resonance frequency F is measured. At step 908, time t is compared to the duration T1 of a specified time interval until time t exceeds T1. When time t exceeds T1, a slope F′ is calculated at step 910. In some embodiments, F′ may be calculated by the equation F′=(F₀−F_{T1})/T1, where F₀ is the resonance frequency measured at time t=0, and F_{T1} is the resonance frequency measured at time t=T1. At step 912, the threshold value (F′ threshold) is determined. In some embodiments, F′ threshold is proportional to F₀. For example, in some embodiments, F′ threshold is equal to F₀ multiplied by a coefficient A. In some embodiments, F′ threshold is calculated from an absolute value of the resonance frequency and updated every T1 seconds.

At step 914, F′ is compared to F′ threshold. If F′ does not exceed F′ threshold, time t is reset to 0 at step 916, and the process returns to step 908 to begin a new time interval with a duration of T1 seconds followed by updated calculations of F′ and F′ threshold. If, at step 914, F′ exceeds F′ threshold, the process proceeds to step 918 which indicates that the incision has been completed. Once completion of the incision has been detected, the ultrasonic treatment may automatically cease operation.

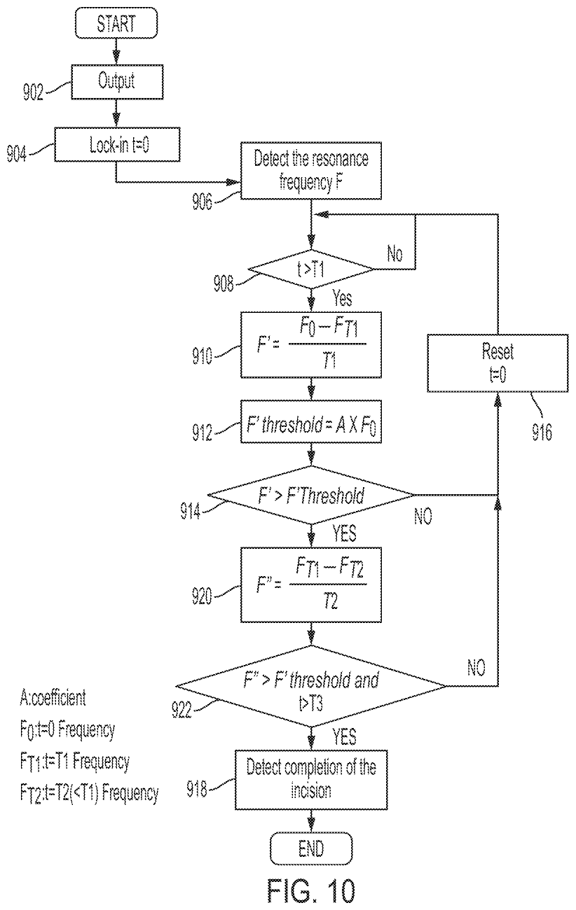

FIG. 10 is a flow chart illustrating a modified version of the process shown in FIG. 9 according to certain embodiments. The process shown in FIG. 10 may be similar to the process shown in FIG. 9, except that following step 914, if F′ is greater than F′ threshold, a second slope F″ is calculated at step 920 and compared to F′ threshold. In some embodiments, F″ may be calculated as F″=(F_{T2}−F_{T1})/T2, where F_{T1} is the resonance frequency measured at time t=T1, and F_{T2} is the resonance frequency measured at time t=T2. If F″ exceeds F′ threshold and time t is greater than a certain time T3, the completion of the incision is detected (step 918). Otherwise the process returns to step 916 where time t is reset to 0, and back to step 908 to start another time interval.

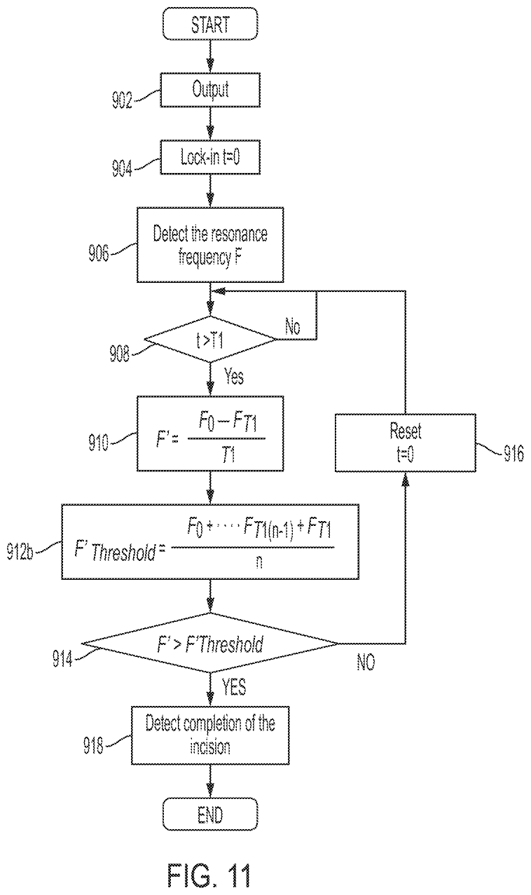

The threshold value F′ threshold is not necessarily limited to the embodiments shown in FIGS. 9 and 10. F′ threshold may be based on other calculations. In some embodiments, for example, F′ Threshold may be an average value of the resonance frequency. FIG. 11 shows a flow chart that is similar to the flow chart of FIG. 9, except that F′ threshold calculated at step 912b is an average value of resonance frequency. In some such embodiments, F′ Threshold may be calculated using the following equation:

$${F}_{Threshold}^{′} = \frac{{F}_{0} + … {F}_{T 1 ( n - 1 )} + {F}_{T 1}}{n}$$

where n is the number of measurement samples during the time interval.

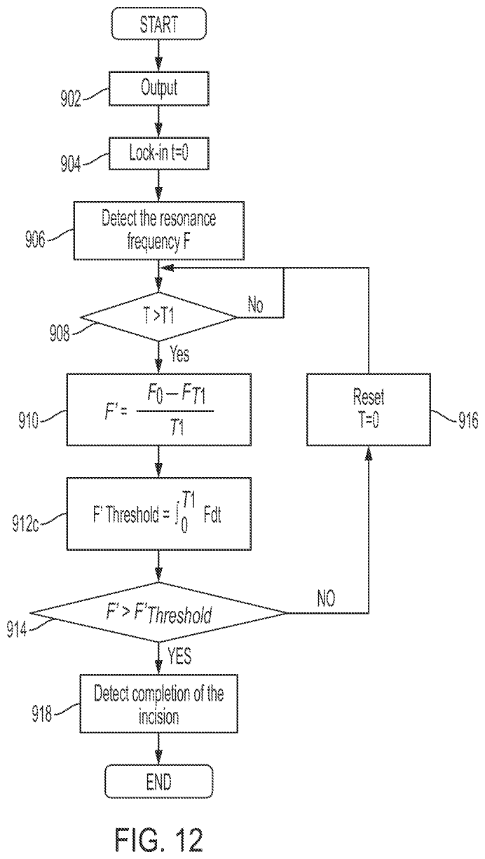

In other embodiments, F′ Threshold may be an integral value of the resonance frequency. FIG. 12 shows a flow chart that is similar to the flow chart of FIG. 9, except that F′ Threshold calculated at step 912c is an integral value of resonance frequency. In some such embodiments, F′ threshold may be calculated using the following equation:

F′Threshold=∫₀^{T1}Fdt

An ultrasonic treatment device according to embodiments of the present disclosure may be configured to detect the completion of an incision using any one or more of the methods described herein and automatically cease operation of the ultrasonic transducer when the completion of the incision has been detected. In some embodiments, for example, a supply of electric energy to the transducer may be automatically stopped or reduced in response to detecting the completion of the incision. In some embodiments, an ultrasonic treatment device may include, for example, a control unit for operating the ultrasonic transducer that is configured to implement one or more of the methods described herein for detecting the completion of an incision. The control unit may include one or more processors for controlling operation of the ultrasonic transducer in accordance with one or more of the described methods. The control unit may further include memory (e.g., one or more nonvolatile storage devices or other non-transitory computer-readable storage mediums) for storing programs, modules, data structures, or a subset thereof, for the one or more processors to control and run the various components and methods disclosed herein. In some embodiments, an ultrasonic treatment device includes a storage medium (e.g., non-transitory computer-readable medium) having stored thereon computer-executable instructions which, when executed by a processor, perform one or more of the methods disclosed herein.

While certain embodiments of the present disclosure have been described in connection with certain instruments and treatment procedures, embodiments described herein are not necessarily limited to these specific uses. It should be understood that various changes, substitutions, and alterations can be made herein without departing from the spirit and scope of the invention as defined by the appended claims. It should also be apparent that individual elements identified herein as belonging to a particular embodiment may be included in other embodiments of the invention. Moreover, the scope of the present application is not intended to be limited to the particular embodiments of the process, machine, manufacture, and composition of matter, means, methods and steps described in the specification. As one of ordinary skill in the art will readily appreciate from the disclosure herein, processes, machines, manufacture, composition of matter, means, methods, or steps, presently existing or later to be developed that perform substantially the same function or achieve substantially the same result as the corresponding embodiments described herein may be utilized according to the present invention.

What is claimed is:

1. A treatment device for treating a target tissue comprising:
a drive source having a transducer configured to convert electrical energy to mechanical vibrations;
an instrument having a blade connected to the drive source and configured to apply the mechanical vibrations to the target tissue, and
a control unit configured to control a supply of electrical energy to the drive source, the control unit being configured to adjust the supply of electrical energy to the drive source in response to a detection of a change in a resonance frequency of the blade.

2. The treatment device of claim 1, wherein the control unit is configured to automatically reduce or stop the supply of electrical energy to the drive source in response to the detection of the change in the resonance frequency of the blade.

3. The treatment device of claim 1, wherein the detection of the change in the resonance frequency of the blade comprises detecting a change from a first trend in the resonance frequency over time to a second trend in the resonance frequency over time.

4. The treatment device of claim 3, wherein the first trend is a decreasing trend in the resonance frequency, and the second trend is an increasing trend in the resonance frequency.

5. The treatment device of claim 1, wherein the detection of the change in the resonance frequency of the blade comprises detecting an occurrence of a peak value of the resonance frequency.

6. The treatment device of claim 1, wherein the control unit is configured to determine a slope of the resonance frequency over a predetermined time period, determine a threshold value, and compare the slope to the threshold value.

7. The treatment device of claim 6, wherein the control unit is configured to update the slope and the threshold value every predetermined time period.

8. The treatment device of claim 6, wherein the threshold value is proportional to the resonance frequency at a specified time.

9. The treatment device of claim 8, wherein the threshold value for a given predetermined time period is a product of the resonance frequency at a start of the given predetermined time period and a coefficient.

10. The treatment device of claim 6, wherein the threshold value for a given predetermined time period is equal to an average resonance frequency over the given predetermined time period.

11. The treatment device of claim 6, wherein the threshold value for a given predetermined time period is equal to an integral of the resonance frequency over the given predetermined time period.

12. The treatment device of claim 1, wherein the control unit is configured to, for each time interval in a series of time intervals:
determine a slope of the resonance frequency over an immediately preceding time interval;
calculate a projected value for the resonance frequency from the slope of the resonance frequency; and
compare the resonance frequency to the projected value for the resonance frequency.

13. The treatment device of claim 12, wherein comparing the projected value for the resonance frequency to the resonance frequency comprises calculating a difference between the resonance frequency and the projected value for the resonance frequency.

14. The treatment device of claim 13, wherein the control unit is further configured to compare a magnitude of the difference to a threshold value.

15. The treatment device of claim 14, wherein the detection of the change in the resonance frequency of the blade comprises detecting that the magnitude of the difference exceeds the threshold value, and
wherein if the magnitude of the difference does not exceed the threshold value within a time interval, the control unit is configured to proceed to the next time interval in the series of time intervals.

16. The treatment device of claim 1, wherein the control unit is configured to adjust the supply of electrical energy to the drive source in response to the detection of the change in the resonance frequency and a change in ultrasonic impedance.

17. A method for controlling a treatment device by using a control unit, the method comprising:
generating mechanical vibrations using a transducer;
transmitting the mechanical vibrations to a blade connected to the transducer;
measuring a resonance frequency of the blade over time; and
stopping the generation of the mechanical vibrations by the ultrasonic transducer in response to detecting a change in the resonance frequency of the blade by the control unit.

18. The method of claim 17, further comprising, for each time interval in a series of time intervals:
calculating a slope of the resonance frequency over the time interval,
calculating a threshold value; and
comparing the slope to the threshold value.

19. The method of claim 17, further comprising, for each time interval in a series of time intervals:
determining a slope of the resonance frequency over an immediately preceding time interval;
calculating a projected value for the resonance frequency from the slope of the resonance frequency; and
comparing the resonance frequency to the projected value for the resonance frequency.

20. The method of claim 19, wherein comparing the projected value for the resonance frequency to the resonance frequency comprises calculating a difference between the resonance frequency and the projected value for the resonance frequency, and
wherein detecting the change in the resonance frequency of the blade comprises detecting that a magnitude of the difference between the resonance frequency and the projected value for the resonance frequency exceeds a threshold value.
